(19) United States
(12) Patent Application Publication
(10) Pub. No.: US 2005/0161609 A1
(43) Pub. Date: Jul. 28, 2005

(54) X-ray detector module for spectrally resolved measurements

(76) Inventors: Bjoern Heismann, Erlangen (DE); Quirin Spreiter, Erlangen (DE); Stefan Wirth, Erlangen (DE)

Correspondence Address: HARNESS, DICKEY & PIERCE, P.L.C., P.O.BOX 8910, RESTON, VA, 20195, US

(21) Appl. No.: 11/035,081

(22) Filed: Jan. 14, 2005

(30) Foreign Application Priority Data
Jan. 16, 2004 (DE) 10 2004 002 463.4
Feb. 10, 2004 (DE) 10 2004 006 547.0

(51) Int. Cl. G01T 1/24; G01N 23/00; G01J 1/00; H05G 1/60; G21K 1/12; A61B 6/00

(52) U.S. Cl. 250/370.09

(57) ABSTRACT

An X-ray detector-module includes one or more rows of detector elements arranged above one another in at least two layers. First detector elements of an upper layer, facing incoming X-radiation, are sensitive to first spectral components of the X-radiation and at least partially transparent to second spectral components of the X-radiation. Second detector elements of a lower layer arranged therebelow, are sensitive to the second spectral components. In the case of the present X-ray detector module, the second detector elements have a larger detector surface than the first detector elements. Further, the ratio and the mutual arrangement of the detector surfaces of in each case a first detector element and a second detector element arranged therebelow is selected such that the first detector element and the second detector element detect the same solid angle of the X-radiation emanating from an X-ray focus with a permanently prescribed relative position in relation to the X-ray detector module.

The present application hereby claims priority under 35 U.S.C. §119 on German patent application numbers DE 10 2004 002 463.4 filed Jan. 16, 2004 and DE 10 2004 006 547.0 filed Feb. 10, 2004, the entire contents of each of which are hereby incorporated herein by reference.

### FIELD OF THE INVENTION

The present invention generally relates to an X-ray detector module. In particular, it relates to an X-ray detector module for computed tomography units, that has one or more rows of detector elements arranged above one another in at least two layers, first detector elements of an upper layer, facing incoming X-radiation, being sensitive to first spectral components of the X-radiation and at least partially transparent to second spectral components of the X-radiation, second detector elements of a lower layer arranged therebelow, being sensitive to the second spectral components, and the first detector elements forming pairs of detector elements with the second detector elements respectively lying therebelow.

### BACKGROUND OF THE INVENTION

At present, use is made in X-ray computed tomography (CT) of single-row or multi-row detector systems that are formed from a large number of individual detector elements. An X-ray detector for computed tomography is generally assembled from a number of individual detector modules that respectively comprise a smaller group of detector elements. Each detector element constitutes one channel of the X-ray detector.

The detector elements measure the intensity of an X-ray beam attenuated after passing through an examination area of an object. The attenuation of the X-ray beam is caused by the trans-irradiated materials along the beam path, and so the attenuation can also be understood as a line integral over the attenuation coefficient of all voxels along the beam path. It is possible to use so-called reconstruction methods to calculated backward from the projected attenuation data to the attenuation coefficients g of the individual voxels.

In the case of recent techniques of computed tomography, spectral information relating to attenuated X-ray beams are also being utilized in order, for example, also to obtain in addition to the spatial distribution of the attenuation coefficients, a distribution of the density and of the effective atomic number inside the examination area. Such a method is disclosed, for example, by German patent application 101 43 131 A1.

However, the use of the spectral information requires the recording of two measured data records with a different spectral distribution of the incident X-radiation or different spectral sensitivity of the detectors. As a rule, a method is used in which the object is irradiated successively with X-radiation of different energy in order to obtain the two measured data records. This leads to a lengthened scanning time, and to the problems associated therewith of the increased exposure to X-rays of the patient, as well as to a possible movement artifact that can occur owing to movement of the patient between two recordings.

In order to avoid these problems, U.S. Pat. No. 4,247,774 proposes an X-ray detector having a number of detector elements, that are arranged in rows in two layers one above another. The detector elements arranged in the upper layer are sensitive to another spectral component of the X-radiation than the detector elements lying therebelow. A pair of detector elements including an upper first detector element and a lower second detector element absorbs only X-ray components of low energy in the upper detector element, which is formed by a thin scintillator crystal with a coupled photomultiplier, whereas the components of higher energy are not absorbed and strike the second detector element. The second detector element has a correspondingly thicker scintillator crystal to which a photomultiplier is likewise coupled, and converts the remaining components of higher energy into an appropriate measurement signal.

Consequently, the detector elements of each of the two layers supply measured data of different spectral weight from which the above information can be derived. DE 198 26 062 A1 describes such an arrangement for detecting X-rays, and in this case a further layer of detector elements is additionally introduced. X-ray detectors of the two publications respectively include a multiplicity of pairs or triplets, respectively, of detector elements that are of identical design.

### SUMMARY OF THE INVENTION

An object of an embodiment of the present application includes providing X-ray detector modules for the construction of an X-ray detector, in particular for computed tomography units that supply measured data of different spectral weight and offer improved usage of the X-radiation by comparison with known X-ray detectors.

An object may be achieved with the aid of an X-ray detector module and/or an X-ray detector. Other advantageous refinements of the X-ray detector module and the X-ray detector can be gathered from the following description and the exemplary embodiments.

The X-ray detector module of one embodiment includes one or more rows of detector elements arranged above one another in at least two layers. First detector elements of an upper layer facing incoming X-radiation are sensitive to first spectral components of the X-radiation and at least partially transparent to second spectral components of the X-radiation. Second detector elements of a lower layer arranged therebelow are sensitive to the second spectral components.

The first detector elements form pairs of detector elements with the second detector elements respectively lying therebelow. In the case of the present X-ray detector module, the second detector elements have a larger detector surface than the first detector elements, the ratio and the mutual arrangement of the detector surfaces of the first detector element and second detector element of each pair of detector elements being selected such that the first detector element and the second detector element of the pair of detector elements detect the same solid angle of the X-radiation emanating from an X-ray focus with a prescribed relative position in relation to the detector module.

The present X-ray detector module permits the simultaneous acquisition of the measured data in terms of two different spectral regions during a single X-ray irradiation, for example, during a single scan with the aid of an X-ray computed tomograph. It is thereby possible, on the one hand, to avoid artifacts that can occur when acquiring the measured data of different spectral weight with the aid of two separate X-ray images. On the other hand, it is also possible to achieve a substantial dose reduction, since only a single CT image is required in order to obtain the two measured data records.

By summing the measurement signals of the respective detector elements of each pair of detector elements, it is also possible to reconstruct a conventional CT image. It is therefore possible to produce a conventional and spectrally resolved image, or distributions derived therefrom, from one and the same scan. The particular arrangement of the detector elements in the individual pairs of detector elements of the detector module produces an optimum utilization of the incident X-radiation, since the solid angles of the incident X-radiation that are detected by the detector elements of each pair of detector elements that lie one above another are identical for the two detector elements.

The individual X-ray detector modules are in this case assembled to form an X-ray detector in such a way that the module-specific relative positions of the X-ray focus correspond to one and the same position in the X-ray detector. In the case of a detection surface of the X-ray detector that is designed to be substantially flat or only slightly curved. This can require the outer detector modules to have a different geometrical design than the inner modules.

Given an arrangement of the individual modules on a polygonal curve aligned with the X-ray focus, all the detector modules can be of the same design with a flat detection surface. The individual detector modules are therefore aligned in the X-ray detector with a common focus, the focus of the X-ray source. It is preferred, in addition, to apply a collimator for X-radiation to the upper layer of the detector modules or of the X-ray detector, or to fit it thereover.

In an advantageous refinement, the present X-ray detector modules of an embodiment are constructed such that they can be implemented without being converted into the data acquisition system (DAS) of an existing 2-row CT unit. The X-ray detector modules are designed in one row for this purpose, the number of detector pairs being selected such that it corresponds to half the number of the detector elements of the 2-row X-ray detector modules of the CT unit. Existing electronics can be used in this way, since no further measuring channels are added.

The first detector elements of the single-row X-ray detector modules are designed with particular advantages such that the width of their detector surfaces in the row direction corresponds to the width of the detector surfaces of the detector elements of the two-row X-ray detector modules, and the extent of their detector surfaces perpendicular to the row direction corresponds to the two-fold extent of the detector surfaces of the detector elements of the conventional two-row X-ray detector modules in the same direction. This configuration permits the present X-ray detector modules to be inserted into the housing designed for the two-row X-ray detector modules without mechanical adaptation. It is thereby possible for already existing computed tomography units to be equipped or retrofitted with the present detector modules in a very simple and cost-effective way, for example, as an option or upgrade.

The two layers of each detector module are preferably produced separately so that it is firstly possible for them to be qualified separately in terms of their image-relevant properties. The quantitative data of the qualification are used to form module pairs that are optimally compatible with reference to these data. After this qualification and pairing, the two components are mechanically adjusted to one another and connected to a collinator to form a unit.

In a further qualification of the connected module units, the spectral sensitivities of the two layers, in particular, are quantitatively checked. These quantitative data are used to assemble an assortment of the module units that ensures a homogeneous image quality of the overall detector—both in each layer individually, and in the total signal from the two layers. Use can be made for this purpose of a method such as is known from DE 198 11 044 C1 for example.

If required the present detector module can, of course, also be constructed from more than two layers of detector elements. In this case the size of the detector surfaces and the mutual arrangement of the individual detector elements are then likewise selected such that the condition with regard to the same solid angle is met.

The detector elements of the present detector module of an embodiment preferably include a scintillator crystal and a photodiode on a module carrier. In order to design the detector elements lying one above another with different spectral sensitivities, the scintillator crystal of the upper detector element can, for example, be of a thinner configuration and/or can include another material than that of the lower detector element. Fundamentally, all materials known for converting X-radiation come into consideration here as scintillator crystals in the way they have already been used in known X-ray detectors.

Furthermore, the shape of the individual detector layers can be selected in accordance with the prior art such that these layers run in a plane, or have a shape curved toward the X-ray source.

### BRIEF DESCRIPTION OF THE DRAWINGS

The present X-ray detector module is explained in more detail once more below with the aid of an exemplary embodiment in conjunction with the drawings, in which:

FIG. 1 shows a schematic illustration of a single-row detector of a computed tomography unit;

FIG. 2 shows an example of a two-row X-ray detector module in accordance with the prior art;

FIG. 3 shows two examples of an X-ray detector module, in accordance with an embodiment of the present invention;

FIG. 4 shows a section through the two layers of the detector modules of FIG. 3; and

FIG. 5 shows an example of the construction of a pair of detector elements of an embodiment of the present detector module.

### DETAILED DESCRIPTION OF THE EXEMPLARY EMBODIMENTS

FIG. 1 shows a schematic of an example of the design of an X-ray detector in a computed tomography unit. The figure shows a single-row detector in the case of which individual detector elements 1 are combined to form modules 2. The detector elements 1 are arranged on a curved surface about the X-ray focus 3 formed by the X-ray source, such that the fan-shaped X-ray beam 4 emanating from the X-ray focus 3 strikes the surface of the detector elements 1 virtually perpendicularly.

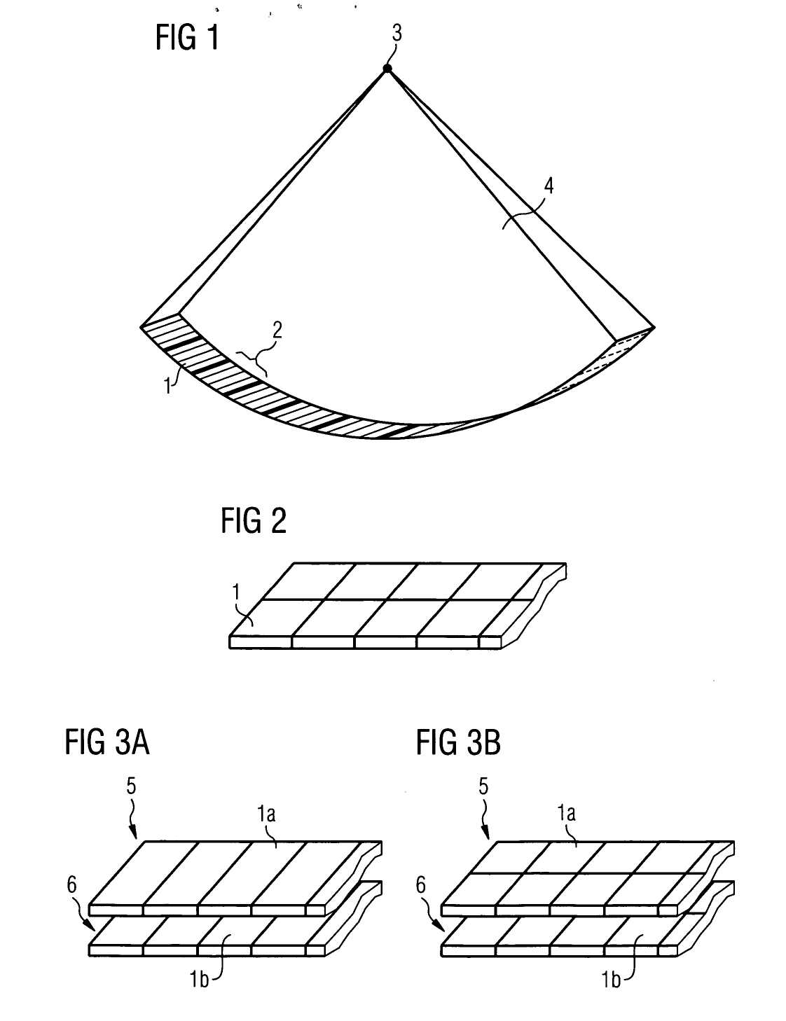

FIG. 2 shows a detector module for a 2-row CT unit in accordance with the prior art. In such a detector module 2, the active surface is generally subdivided into 2×16 individual segments in the form of the detector elements 1, 16 detector elements in the direction of rotation of the computer tomograph and 2 rows in the direction of the patient axis, in order to be able to record two object layers simultaneously during one rotation of the gantry.

In one embodiment of the present detector module, the respectively 16 detector elements are now no longer arranged in the example of FIG. 3a in 2 rows next to one another, but in two layers one behind another. The detector surfaces of the upper detector elements 1a have been enlarged, as may be seen from FIG. 3a, such that a detector element 1a covers the previous 2 rows. In this way, the number of channels of the detector module remains the same by comparison with the two-row detector module of FIG. 2.

FIG. 3b shows a further possibility of configuring the detector module 2 as a two-row detector that has twice the number of detector elements by comparison with the detector of FIG. 2.

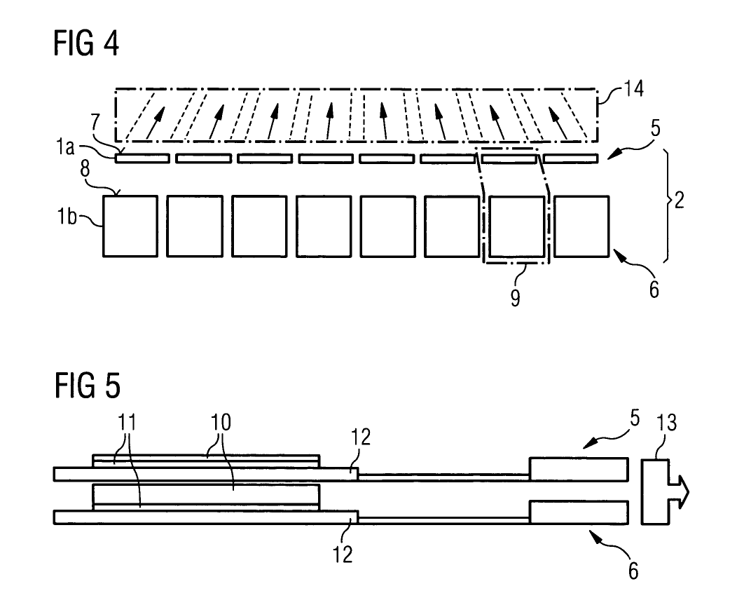

Since, by comparison with the upper layer 5 of the detector elements 1a, 1b, the lower layer 6 has a greater distance from the X-ray focus, the detector elements 1b of the lower layer 6 have a correspondingly greater detector surface for covering the same solid angle. This is illustrated with the aid of FIG. 4, which shows a section through the detector elements 1a, 1b of the two layers 5, 6 of a detector module 2.

Here, the arrows indicate the direction to the focus of the X-ray tube of the computer tomograph. The individual detector elements of the upper layer 5 and the lower layer 6 are mutually arranged in this case in such a way, and particularly partly displaced relative to one another, and the detector elements 1b of the lower layer 6 have a correspondingly larger detector surface 8 than those of the upper layer 5, that the detector elements 1a, 1b of each pair 9 of detector elements of the detector module 2 detect the same solid angle of the X-radiation emanating from the focus. Furthermore, FIG. 4 indicates by dashes a collimator 14 (not true to scale) that can be applied to the detector module 2.

FIG. 5 shows a schematic of the design of a pair 9 of detector elements in accordance with an exemplary embodiment of the present invention. Each layer is constructed in this case firstly as a dedicated component consisting of a scintillator 10, a photodiode 11 and module carrier 12. Used as the upper layer 5 facing the X-ray source is a scintillator array that is thinned down by additional process steps to 200 μm from an array of standard thickness. The correspondingly thinner scintillator crystal 10 by comparison with the lower layer 6 is clearly evident in the figure. The low-energy part of the X-ray spectrum is chiefly absorbed in the upper layer 5, and converted into light. The higher-energy X-ray quanta penetrate the upper layer 5 and are overwhelmingly absorbed in the lower layer 6. This results in a different spectral sensitivity of these two layers.

The measurement signals generated by the photodiodes 11 from the two layers 5, 6 are regrouped by an adapter in an example such that they can be further processed and evaluated by the DAS 13. In the case of a refinement of the module in accordance with FIG. 3a, there is no need here for any further mechanical or electrical modifications of a standard system that was configured for a 2-row detector in accordance with FIG. 2.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. An X-ray detector module, comprising:
at least one row of detector elements arranged above one another in at least two layers, wherein first detector elements of a relatively upper layer, facing incoming X-radiation, are sensitive to first spectral components of the X-radiation and at least partially transparent for second spectral components of the X-radiation, and wherein second detector elements of a relatively lower layer arranged therebelow, are sensitive to the second spectral components, the first detector elements forming pairs of detector elements with the second detector elements respectively lying therebelow, wherein the second detector elements have a relatively larger detector surface than the first detector elements, and wherein the ratio and the mutual arrangement of the detector surfaces of the first detector element and second detector element of each pair of detector elements are selected such that the first detector element and the second detector element of the pair of detector elements detect the same solid angle of the X-radiation emanating from an X-ray focus with a permanently prescribed relative position in relation to the X-ray detector module.

2. The X-ray detector module as claimed in claim 1, wherein the detector elements each include a scintillator crystal and a photodiode, arranged on a module carrier.

3. The X-ray detector module as claimed in claim 2, wherein the first detector elements have a relatively thinner scintillator crystal than the second detector elements.

4. The X-ray detector module as claimed in claim 1, wherein a collimator, aligned with the X-ray focus, is arranged at least one of over and on the upper layer.

5. An X-ray detector having a number of X-ray detector modules as claimed in claim 1, wherein the number of X-ray detector modules are arranged next to one another and are constructed such that an X-ray focus position prescribed for the X-ray detector responds to an X-ray source at each X-ray detector module of the X-ray detector with the prescribed relative position.

6. A computed tomography unit including a single row of X-ray detector modules as claimed in claim 1, wherein a number of detector pairs of the individual X-ray detector modules is selected to correspond to half the number of the detector elements of conventional two-row X-ray detector modules of a computed tomography unit.

7. The computed tomography unit as claimed in claim 6, wherein the first detector elements of the single-row X-ray detector modules are designed such that an extent of their detector surfaces in the row direction corresponds to an extent of detector surfaces of the detector elements of the conventional two-row X-ray detector modules in the same direction, and an extent of their detector surfaces perpendicular to the row direction corresponds to a two-fold extent of the detector surfaces of the detector elements of the conventional two-row X-ray detector modules in the same direction, such that the single-row X-ray detector modules is insertable without mechanical adaptation into a housing designed for the two-row X-ray detector modules.

8. The X-ray detector module of claim 1, wherein the X-ray detector module is for a computed tomography unit.

9. A computed tomography unit, comprising the X-ray detector module of claim 1.

10. The X-ray detector module as claimed in claim 2, wherein a collimator, aligned with the X-ray focus, is arranged at least one of over and on the upper layer.

11. The X-ray detector module as claimed in claim 3, wherein a collimator, aligned with the X-ray focus, is arranged at least one of over and on the upper layer.

12. An X-ray detector having a number of X-ray detector modules as claimed in claim 2, wherein the number of X-ray detector modules are arranged next to one another and are constructed such that an X-ray focus position prescribed for the X-ray detector responds to an X-ray source at each X-ray detector module of the X-ray detector with the prescribed relative position.

13. An X-ray detector having a number of X-ray detector modules as claimed in claim 3, wherein the number of X-ray detector modules are arranged next to one another and are constructed such that an X-ray focus position prescribed for the X-ray detector responds to an X-ray source at each X-ray detector module of the X-ray detector with the prescribed relative position.

14. An X-ray detector having a number of X-ray detector modules as claimed in claim 4, wherein the number of X-ray detector modules are arranged next to one another and are constructed such that an X-ray focus position prescribed for the X-ray detector responds to an X-ray source at each X-ray detector module of the X-ray detector with the prescribed relative position.

15. An X-ray detector having a number of X-ray detector modules as claimed in claim 8, wherein the number of X-ray detector modules are arranged next to one another and are constructed such that an X-ray focus position prescribed for the X-ray detector responds to an X-ray source at each X-ray detector module of the X-ray detector with the prescribed relative position.
